(19) United States
(12) Patent Application Publication
(10) Pub. No.: US 2026/0327177 A1
(43) Pub. Date: Oct. 22, 2026

(54) Method and System for Producing Ethylene and Acetic Acid, and Method and System for Producing a Target Compound Using Ethylene and Acetic Acid

(71) Applicant: Linde GmbH, Pullach (DE)

(72) Inventors: Andreas Meiswinkel, Rimsting (DE); Martin Schubert, Pullach (DE); Desislava Tota, Munchen (DE); Mathieu Zellhuber, Martinsried (DE)

(21) Appl. No.: 19/114,778

(22) PCT Filed: Sep. 26, 2023
(86) PCT No.: PCT/EP2023/076555

§ 371 Date: Mar. 24, 2025

(30) Foreign Application Priority Data
Sep. 26, 2022 (EP) 22020458.0

(51) Int. Cl. C07C 51/25; C07C 5/48

(52) U.S. Cl. CPC C07C 51/25; C07C 5/48; C07C 2527/057

(57) ABSTRACT

A process for preparing ethylene and acetic acid includes subjecting ethane and oxygen in a feed mixture to oxidative catalytic dehydrogenation (OCD) to obtain a product mixture containing ethylene, acetic acid and further components. Ethylene in the feed mixture is formed by ethylene contained in the product stream which is recycled to the OCD. The product mixture is subjected to a primary treatment, from which a subsequent mixture is taken which is depleted in carbon dioxide and water compared to the product mixture. The subsequent mixture is fed as a cryogenic separation feed to a cryogenic separation which comprises a demethanization and an ethane-ethylene separation. The separation feed is fed to the demethanization as a demethanization feed. During demethanization a heavy fraction and a light fraction are formed as an overhead fraction. The heavy fraction is fed to the ethane-ethylene separation, and the light fraction is recycled to the OCD.

### CROSS-REFERENCE TO RELATED APPLICATIONS

This application is the national phase of, and claims priority to, International Patent Application No. PCT/EP2023/076555, filed 26 Sep. 2023, which claims priority to European Patent Application No. EP22020458.0, filed 26 Sep. 2022.

### FIELD OF THE INVENTION

The invention relates to a process and a plant for preparing ethylene and acetic acid, and to a process and a plant for preparing a target compound using ethylene and acetic acid.

### BACKGROUND OF THE INVENTION

The oxidative dehydrogenation (ODH) of paraffins with two to four carbon atoms is known in principle. In ODH, the paraffins mentioned are reacted with oxygen to form, among other things, the respective olefins and water. The invention relates to the oxidative dehydrogenation of ethane to ethylene, hereinafter also referred to as ODHE. Therefore, when reference is made to “oxidative catalytic dehydrogenation” below, this is understood in particular to mean oxidative catalytic dehydrogenation of ethane.

ODHE may have advantages over more established processes for producing olefins, such as steam cracking. Due to the exothermicity of the reactions involved and the practically irreversible water formation, there is no thermodynamic equilibrium limitation. The ODHE can be carried out at comparatively low reaction temperatures. In principle, no regeneration of the catalysts used is necessary, since the presence of oxygen enables or causes in-situ regeneration. Finally, in contrast to steam cracking, smaller amounts of worthless by-products, such as coke, are formed.

For further details regarding ODHE, reference is made to relevant literature, for example Ivars, F. and López Nieto, J. M., Light Alkanes Oxidation: Targets Reached and Current Challenges, in: Duprez, D. and Cavani, F. (eds.), Handbook of Advanced Methods and Processes in Oxidation Catalysis: From Laboratory to Industry, London 2014: Imperial College Press, pages 767-834, or Gartner, C. A. et al., Oxidative Dehydrogenation of Ethane: Common Principles and Mechanistic Aspects, ChemCatChem, Vol. 5, No. 11, 2013, pages 3196 to 3217, as well as X. Li, E. Iglesia, Kinetics and Mechanism of Ethane Oxidation to Acetic Acid on Catalysts Based on Mo—V—Nb Oxides, J. Phys. Chem. C, 2008, 112, 15001-15008.

US 2002/082445 A1 discloses a process for the oxidation of a C2 to C4 alkane to produce the corresponding alkene and the corresponding carboxylic acid, the process comprising contacting the alkane, a molecular oxygen-containing gas and the corresponding alkene and optionally water in an oxidation reaction zone in the presence of at least one catalyst that is active for the oxidation of the alkane to the corresponding alkene and the carboxylic acid to produce a product stream comprising the alkene, the carboxylic acid and water. In the process, the molar ratio of alkene to carboxylic acid produced in the oxidation reaction zone is adjusted to or maintained at a predetermined value by controlling the concentrations of the alkene and optionally water in the oxidation reaction zone and optionally also one or more of the factors of pressure, temperature and residence time of the oxidation reaction zone. Such an oxidation process can be used in an integrated process, for example for the production of vinyl acetate or ethyl acetate.

In particular, MoVNb-based catalyst systems have proven to be promising for ODHE, as described for example in F. Cavani et al., “Oxidative dehydrogenation of ethane and propane: How far from commercial implementation?”, Catal. Today, 2007, 127, 113-131. Catalyst systems containing additional Te can also be used. When reference is made here to a “MoVNb-based catalyst system” or a “MoVTeNb-based catalyst system”, this means a catalyst system that contains the elements mentioned as a mixed oxide, also expressed as MoVNbOₓ or MoVTeNbOₓ. The indication of Te in parentheses indicates its optional presence. The invention is used with such catalyst systems.

In ODHE, especially when using MoVNb(Te)Oₓ-based catalysts under industrially relevant reaction conditions, a significant amount of acetic acid is formed as a by-product. Therefore, for economical plant operation, co-production and further use of ethylene and acetic acid is generally unavoidable and necessary when using the catalyst type described. In this case, a preferential formation of ethylene is usually desirable in technical applications, or the formation of acetic acid is undesirable in conventional processes. However, in integrated petrochemical complexes there is often a need for precisely these two value products. Important technical processes exist that require both ethylene and acetic acid as reactants, as explained below. There is therefore a need for ways to produce ethylene and acetic acid, in particular in the ratio required for corresponding processes.

### SUMMARY

A process for the production of ethylene and acetic acid is proposed, in which ethane and oxygen in a feed mixture are subjected to oxidative catalytic dehydrogenation using a catalyst containing at least molybdenum, vanadium and niobium and optionally tellurium as a mixed oxide, to obtain a product mixture containing ethylene, acetic acid and other components.

For corresponding catalysts which can be used in the context of the invention, in particular diluted with corresponding diluent materials or on suitable, in particular inert, supports, reference is made to the above explanations. A corresponding catalyst is in particular sulfur-free or free of sulfur species or has sulfur or sulfur species in a content of less than 100 ppm by weight (micro weight portion), in particular less than 10 ppm by weight, more particularly less than 1 ppm by weight. In the context of the invention, the feed mixture has an ethylene content of 0.25 to 30 mol. % (mol percent), in particular from 0.5 to 25 mol. %, more particularly 1 to 20 mol. %, whereby the content can generally, and regardless of the respective lower limit, be in particular less than 25 mol. %, more particularly less than 20 mol. %, even more particularly less than 15 mol. %.

The proposed process is based, as also explained below, on the surprising finding that by using an appropriate proportion of ethylene in a feed stream for oxidative catalytic dehydrogenation, the proportion of acetic acid in the product stream can be significantly increased when using an appropriate catalyst. In particular, the ratio of ethylene to acetic acid in the product stream can be specifically adjusted thereby, so that it corresponds to the requirements of at least one downstream process or the average requirements of several downstream processes. In this case, this setting can largely correspond to the actual requirements; smaller deviations or fluctuations can be compensated for by suitable intermediate storage facilities (tanks), such as are usually present in any case in a corresponding complex, both for ethylene and for acetic acid.

In the invention, at least a portion of the ethylene content of the feed mixture of the oxidative catalytic dehydrogenation is formed by ethylene which is contained in the product stream of the oxidative catalytic dehydrogenation and is recycled to the oxidative catalytic dehydrogenation. In this way, the ethylene content can be adjusted as required without the use of externally supplied ethylene.

The ethylene content in the feed mixture can be increased or adjusted by various adjustments to the plant operation, in particular in the form of changes to the operating parameters and/or removal of partial streams in the separation part of a corresponding plant and their addition to the feed mixture. In particular, the operational adjustments made only allow the ethylene content in the feed mixture to be significantly increased, while other component contents (in particular ethane) remain approximately the same or are reduced.

In the proposed process, the product mixture or a part thereof is subjected to primary processing, from which a subsequent mixture is taken which is depleted in carbon dioxide and water compared to the product mixture.

The subsequent mixture or a part thereof is fed as a cryogenic separation feed to a cryogenic separation, which enables demethanization and in particular ethane-ethylene separation.

At least a portion of the cryogenic separation feed is fed to the demethanization as a demethanization feed, and in the demethanization a heavy fraction and a light fraction are formed, the latter as the overhead fraction. Compared to the subsequent mixture, the heavy fraction is enriched in compounds or hydrocarbons with two carbon atoms (C2 compounds, in particular a sum of ethane and ethylene) and depleted in the components boiling lower than ethylene, whereas the light fraction is depleted in the compounds with two carbon atoms and enriched in the components boiling lower than ethylene, compared to the subsequent mixture. According to the invention, the light fraction contains a significant amount of ethylene, i.e. 1 to 90 mole or volume percent.

The proposed process may comprise feeding the heavy fraction or a portion thereof to ethane-ethylene separation. However, the process in any case involves the light fraction or a part thereof being recycled to the oxidative dehydrogenation.

In the proposed process, a desired amount of ethylene is discharged via the overhead stream of a modified demethanization together with the usual so-called C1 fraction, which consists of the compounds boiling lower than ethylene. As an additional advantage, a liquid return flow containing ethylene can additionally be ensured, even at comparatively high temperatures at the top of such a modified demethanization. In addition, in the product stream of the catalytic oxidative dehydrogenation, the proportion of a C1 fraction or of compounds boiling lower than ethylene is usually comparatively low. In particular, no or only a small amount of methane (for example a certain proportion originating from the use of ethane in the catalytic oxidative dehydrogenation) is to be expected. Thus, substantially carbon monoxide and ethylene (as the dominant C2 compound) remain in the overhead stream of this modified demethanization. The carbon monoxide is then also returned to the catalytic oxidative dehydrogenation and is at least partially converted into carbon dioxide. Since a significantly increased mass flow now results at the top of a correspondingly modified demethanization, higher oxygen inlet concentrations in the feed stream of the modified demethanization are also permissible, without a critical enrichment of oxygen in the overhead stream occurring. In corresponding embodiments, an effective prevention of reaching explosive compositions (i.e. an oxygen concentration above the oxygen limit concentration, SGK) in the overhead stream of the modified demethanizer by dilution with C2 compounds (in particular ethylene) is nevertheless achieved.

In one embodiment of the invention, a proportion (in particular quantity or volume) of 1% to 35% of the total ethylene contained in the product stream of the oxidative catalytic dehydrogenation can be recycled to the oxidative catalytic dehydrogenation. In particular, lower limits of the proportion of 1%, 2% or 4% and upper limits of 35%, 30%, 20%, 10% and 5% may also be provided. Such values have proven to be particularly favorable, as shown below.

The removal of an ethylene stream or a mixed stream containing ethane and ethylene for recirculation could in principle take place at different points in the process (“removal point”). Accordingly—but not as an exhaustive list—the embodiments explained below can be used, the process proposed here including a removal (also) at the top of a modified demethanization.

In embodiments of the invention, the recycled ethylene can be recycled to the oxidative catalytic dehydrogenation with ethane contained in the product mixture, in particular without separation from one another. This makes it possible, in particular, to recycle corresponding mixed fractions and to avoid the need for complete separation.

The mentioned further components of the product mixture in the oxidative catalytic dehydrogenation can in particular comprise unreacted ethane, compounds boiling lower than ethylene, including in particular carbon monoxide, as well as carbon dioxide and water, the subsequent mixture, which, as mentioned, is formed using a primary processing using the product mixture or a part thereof and is depleted of carbon dioxide and water compared to the product mixture, containing at least a portion of the ethane, the ethylene and the compounds boiling lower than ethylene from the product mixture.

The primary treatment may in particular comprise condensate separation, forming a water-acetic acid fraction, as well as carbon dioxide removal and drying. Furthermore, in particular compression and raw gas treatment (as explained below) are also part of the primary processing. An acetic acid fraction of any purity can be obtained from the water-acetic acid fraction, in particular in an acetic acid processing plant. Corresponding processing steps of the primary processing can be designed in a standard manner and in particular represent a “warm” part of a corresponding separation sequence.

One embodiment of the invention can in particular comprise carrying out a raw gas treatment in the primary processing, which in particular represents a catalytic removal of acetylenes, as well as a carbon dioxide removal, in particular in the form of an amine wash, and and in particular adsorptive drying, a part of a gas mixture taken from the raw gas treatment being able to be carried out upstream of the carbon dioxide removal and drying from the primary processing and being able to be returned to the oxidative catalytic dehydrogenation. Carbon dioxide, which in this embodiment is still contained in a corresponding gas mixture, can be regarded to a certain extent as inert in the oxidative catalytic dehydrogenation, in particular with the catalyst used here and the typical reaction temperatures. An advantage arises here in particular in an easing of the burden on carbon dioxide removal, as a smaller amount of gas needs to be processed there.

Within the scope of the invention, for the low-temperature separation, which, as mentioned, comprises a demethanization and an ethane-ethylene separation, different embodiments can be provided, which relate in particular to the order of these steps.

In one embodiment, at least a portion of the low-temperature separation feed can be fed to the demethanization as the demethanization feed in a material composition that is unchanged compared to the subsequent mixture formed in the primary processing, but also optionally after hydrogenation or another catalytic reaction, in particular for the removal of acetylene. The demethanization can be carried out in a conventional manner, but in the proposed process it is modified or operated in such a way that the head fraction or light fraction (the terms are used synonymously here) has a certain ethylene content according to the invention.

This ethylene content (the following FIGURES are each expressed in mole or volume percent) is, according to the invention, 1 to 90%, in particular 5 to 75%, for example 10 to 50%. As a further component, in a list that is not necessarily exhaustive, in particular carbon monoxide is contained in a content of up to 75%, in particular up to 50%, more particularly up to 25%, for example up to 10%, but only little or no methane, i.e. a methane content is in particular up to 15%, more particularly up to 10%, even more particularly up to 5%, for example up to 2%. Ethane may also be present, with corresponding contents ranging from 0 to 50%, in particular from 0 to 30%, and more particularly from 0 to 20%. It goes without saying that the values mentioned can in each case add up to 100%. Oxygen is present in particular at a content below the SLC, in particular below 5, 2.5, 1 or 0.5%.

This head fraction is therefore recycled to the oxidative dehydrogenation in the proposed process. At least ethane can also be recycled to the oxidative dehydrogenation from the bottom fraction or heavy fraction (these terms are also used synonymously here) of the demethanization. In particular, in this case, a C2 splitter of known type can also be interposed, i.e. an at least partial separation of ethane and ethylene.

An ethane-rich fraction obtained here can then be recycled in particular to the oxidative dehydrogenation.

In alternative embodiments, the order of demethanization and ethane-ethylene separation is reversed. In this case, at least a portion of the low-temperature separation feed, in particular in a material composition unchanged compared to the subsequent mixture formed in the primary processing, but also optionally after hydrogenation or another catalytic reaction, in particular for the removal of acetylene, can be fed to the ethane-ethylene separation as an ethane-ethylene separation feed, wherein in the ethane-ethylene separation a heavy fraction can be formed which is enriched in ethane and depleted in ethylene and the components boiling lower than ethylene compared to the subsequent mixture formed in the primary processing, and a light fraction can be formed which is depleted in ethane and enriched in ethylene and the components boiling lower than ethylene compared to the subsequent mixture formed in the primary processing, wherein the light fraction or a portion thereof is fed to the demethanization as a demethanization feed. A corresponding embodiment enables advantageous effects, in particular through an adapted feeding of a corresponding ethane-ethylene separation or an adapted removal of component mixtures.

Thus, in a corresponding embodiment, the overhead fraction of the demethanization can contain a portion of the ethylene contained in the subsequent mixture formed in the primary processing, in particular to the extent already mentioned, wherein at least a portion of the overhead fraction can be recycled to the oxidative catalytic dehydrogenation to provide at least a portion of the ethylene content of the feed mixture.

The removal of a mixed stream containing ethane and ethylene outside the ethane-ethylene separation, e.g. a part of a bottom stream from the demethanization, and subsequent mixing into the typically recycled ethane stream from the bottom of an ethane-ethylene separation is also provided. Optionally, the mixed stream and/or the recycled ethane stream can be expanded before mixing and, for example, heated against process gas to be cooled. In such an operating mode, changing the ethylene recycle rate advantageously does not require any changes to the overhead and bottom specifications of the ethane-ethylene separation. There is mainly a change in the load of the ethane-ethylene separation (inlet gas quantity) as well as a moderate shift in the inlet gas composition.

In further embodiments, one or more further fractions selected from the overhead fraction formed in the ethane-ethylene separation and the bottom fraction formed in the ethane-ethylene separation can be at least partially or in each case at least partially recycled to the oxidative dehydrogenation. Corresponding fractions, or recycled portions thereof, may be combined with one another in any suitable manner and in any proportions.

In this case, the one or at least one of the several further fractions, like the overhead fraction formed in the demethanization and the bottom fraction formed in the demethanization, are advantageously formed as or each as a fraction or fractions containing ethane and ethylene. These are mixed fractions that are produced in the manner explained above and may also contain other components, depending on where they are formed, such as oxygen and/or carbon monoxide. If part of a gas mixture is removed from the primary processing as explained above, it may in particular still contain carbon dioxide. However, corresponding pure fractions (ethane and/or ethylene fractions) can also be recycled in a corresponding manner.

In particular, a side stream containing ethane and ethylene can also be taken from the ethane-ethylene separation, which stream is at least partially recycled to the oxidative dehydrogenation.

In this case, in embodiments of the invention, the ethylene slip at the bottom of the ethane-ethylene separation can also be increased by partially reducing the reboiler power. In particular, this makes it possible to achieve higher desired ethylene contents in the reaction feed without further extraction and admixture processes, and possible additional heat exchanger passages for heating ethylene-containing extraction streams are eliminated.

A particular variant here is when the light gas slip is used at the bottom of an ethane-ethylene separation upstream of demethanization, since in this case, in addition to ethylene, the proportion of components boiling lower than ethylene (in particular carbon monoxide and optionally methane) in the feed mixture is at least slightly increased.

In one embodiment of the invention, in particular ethylene can be removed at the top of the ethane-ethylene separation or at any point in an optionally available open ethylene refrigeration circuit into which the ethane-ethylene separation is integrated. The sampling point is determined depending on the desired state of aggregation and the pressure and temperature level of the ethylene stream. Analogously, the addition to the other reaction feed streams takes place as far as possible in a single-phase manner, at a similar pressure and temperature level.

Thus, a portion of the ethylene contained in the product mixture can be transferred to an open refrigerant circuit, wherein at least a portion of the ethylene can be discharged from the open refrigeration circuit and recycled to the oxidative catalytic dehydrogenation to provide at least a portion of the ethylene content of the feed mixture.

The recycling alternatives explained above can be carried out in particular in connection with the raw gas treatment already mentioned as part of the primary processing, i.e. the primary processing steps mentioned can in particular comprise at least a partial reaction of acetylenes and oxygen contained in the product mixture. However, further embodiments of the invention can also comprise hydrogenation downstream of demethanization and upstream of ethane-ethylene separation as well as downstream of ethane-ethylene separation. In the latter cases, i.e. in the case of hydrogenation, in particular what is known as tail-end hydrogenation, oxygen is typically not removed, in contrast to a raw gas treatment, so that it can pass into the recycled gas mixture, in particular in a light gas fraction of a demethanization. In all embodiments, advantageously such recycling is carried out that does not recycle any acetylenes. A person skilled in the art selects corresponding alternatives in an appropriate manner.

In embodiments of the invention, at least one material stream recycled to the oxidative catalytic dehydrogenation to provide at least a portion of the ethylene content of the feed mixture or any other material streams can be subjected to processing, in particular a separation of hydrocarbons with three or more carbon atoms, in order to thereby separate heavier components both from the reaction feed of the catalytic oxidative dehydrogenation and from the recycle stream. If required, such processing can in particular also be carried out together with a fresh feed.

In the case of an ethylene feed, an increased formation of carbon monoxide and carbon dioxide is to be expected in the oxidative catalytic dehydrogenation. Here, the already existing product purification of a corresponding plant for oxidative catalytic dehydrogenation can advantageously be used, which can in particular include demethanization and carbon dioxide removal.

In addition, further purification steps can be used in the process, such as trace removal or raw gas treatment and/or selective hydrogenation of hydrocarbons with two carbon atoms. An acetic acid fraction can be prepared by known processes and, if required, acetic acid can be obtained either in concentrated aqueous solution or in pure form. This thus provides acetic acid for corresponding downstream processes. The ethylene fraction can also be provided as needed and at least in part for these downstream processes. Depending on availability at the site, however, additional ethylene or an ethylene-containing or ethylene-rich stream from other sources, such as a steam cracker, can also be used.

The invention also relates to a process for preparing a target compound using ethylene and acetic acid, wherein a process according to the invention is used to provide at least a portion of the ethylene and the acetic acid. In this case, regarding the advantages and embodiments of the provision of ethylene and acetic acid, reference is made to the above explanations.

By using embodiments of the invention, particularly advantageous ratios of ethylene to acetic acid of less than 5:1, less than 3:1, less than 2:1 or less than 1.5:1 can be achieved for a downstream process (without taking into account the ethylene which is recycled). In particular, the ratio is at least 0.8:1 to 1:1.

The target compound of a corresponding process can in particular be vinyl acetate monomer, ethyl acetate, ethylene vinyl acetate or polyethylene terephthalate. As also explained below, in such processes there is a particular need for ethylene and acetic acid in the proportions that can advantageously be provided by embodiments of the invention.

The invention also relates to a plant which is designed to carry out a process as explained above. For this reason, explicit reference is also made to the above explanations regarding a corresponding plant.

### BRIEF DESCRIPTION OF THE DRAWING

The FIGURE illustrates a process according to an embodiment of the invention in the form of a schematic flow chart.

### DETAILED DESCRIPTION

If reference is made below to aspects of a process, the explanations apply in the same way to corresponding plants and their embodiments. The same applies to process steps and plant components. Process steps and plant components with the same or comparable function and/or technical implementation are indicated with identical reference signs.

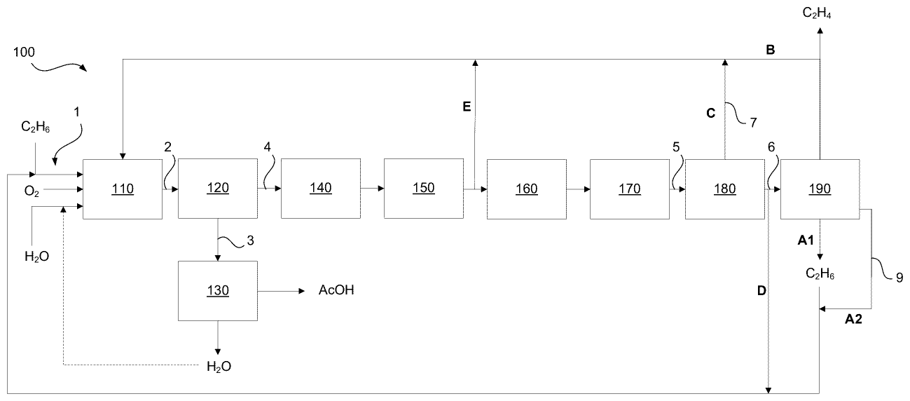

The process illustrated in FIG. 1 corresponds to an embodiment of the invention and is designated overall by 100. In the process 100, the required reactants ethane C₂H₆ and oxygen O₂, which are indicated here as a whole by 1, are supplied to catalytic oxidative dehydrogenation 110. The recycle streams explained below can also be used to provide the corresponding material streams. Additionally, if required, water vapor H₂O can be fed as a diluent into the catalytic oxidative dehydrogenation 110. The water required for this purpose can come at least partly from the raffinate water phase of an acetic acid purification 130 explained below, as illustrated by a dashed arrow.

In the catalytic oxidative dehydrogenation 110, a product mixture 2 is formed, which is subjected to a condensate separation 120. In this, a condensate fraction 3 is formed, which essentially contains water and acetic acid. The condensate fraction 3 is subjected to the mentioned acetic acid treatment 130, in which a water fraction H₂O containing substantially water, and an acetic acid fraction AcOH containing substantially acetic acid, are formed.

Downstream of the condensate separation 120, the product mixture 2, now designated by 4, is present in a form depleted in water and acetic acid. It is then successively subjected to compression 140, optional raw gas treatment 150, carbon dioxide removal 160 and drying 170. Steps 120 to 170 were previously referred to as “primary processing”. Downstream of this there is a component mixture designated by 5, which was previously also called the subsequent mixture. This contains at least a portion of the ethane, the ethylene and the compounds boiling lower than ethylene from the product mixture 4. Compared to product mixture 4, it is depleted in carbon dioxide and water or substantially free of these components.

Then, using at least a portion of the subsequent mixture 5 formed in the primary treatment, a further component mixture is formed in a demethanization 180 which, compared to the subsequent mixture 5, is depleted in the compounds boiling lower than ethylene and enriched in ethane and ethylene. In this case, in the demethanization 180, an overhead fraction 7 and the said further component mixture are formed as a bottom fraction 6, the latter being taken in particular from a column bottom of a column used in the demethanization 180.

Finally, using at least a portion of the bottom fraction 6 in an ethane-ethylene separation 190, also referred to as a C2 splitter, an ethylene fraction C₂H₄, which is enriched in ethylene and depleted in ethane compared to the bottom fraction 6, and an ethane fraction C₂H₆, which is enriched in ethane and depleted in ethylene compared to the bottom fraction 6 of the demethanization 180, are formed, as is usual for an ethane-ethylene separation.

A1, A2, B, C, D and E illustrate recycle streams according to embodiments of the invention. Each recycle stream can, optionally in deviation from the specific representation in FIG. 1, be returned individually or in any combination with other streams.

In this case, A1 denotes the ethane fraction from the ethane-ethylene separation, which is returned to the oxidative catalytic dehydrogenation 110 for further reaction of the starting material ethane. This can, as designated by A2, be combined with a material stream 9 withdrawn via an intermediate take-off of the ethane-ethylene separation 190, for example a rectification column used in the ethane-ethylene separation 190, which has a certain ethylene content which can be adjusted in particular via the withdrawal point, and can be returned together with this to the oxidative catalytic dehydrogenation 110. In general, whenever a combination of different material streams is described before recycling into the oxidative catalytic dehydrogenation 110, a separate recycling is also possible as an alternative.

Material stream B represents a portion of the ethylene fraction from the ethane-ethylene separation 190, which can be recycled to the oxidative catalytic dehydrogenation 110, in particular as an alternative or in addition to the embodiment designated A2. In this case, it can be combined with the overhead fraction 7 from the demethanization 180, as indicated by C. In this case, in particular, in contrast to a conventional demethanization, the demethanization 180 can be operated in such a way that, in addition to components boiling lower than ethylene, a portion of the ethylene also passes into the overhead fraction 7. However, as indicated by D, a portion of the bottom fraction 9 from the demethanization 8, which contains ethane and ethylene, can also be recycled. As also in other cases, any combination of corresponding material streams is possible before recycling.

Material stream E represents a portion of a gas mixture taken from the raw gas treatment 150, which is discharged from the primary processing 120-170 upstream of the carbon dioxide removal 160 and drying 170 and is returned to the oxidative catalytic dehydrogenation 110.

Before explaining specific aspects of the invention and its embodiments, the principles of the invention and further aspects are explained again below for classification.

As mentioned, especially when using MoVNbTeOₓ catalysts in oxidative catalytic dehydrogenation under industrially relevant reaction conditions, acetic acid results as a co-product in addition to ethylene. In this case, under industrially relevant conditions, ethylene to acetic acid ratios of approximately 5 to 10 mol/mol are typically achieved. However, the demand for acetic acid is often limited to specific downstream products (e.g. vinyl acetate monomer). Acetic acid can be separated as a component of the aqueous condensate phase (typical acetic acid content in the condensate: 1 to 30 wt. %, 3 to 25 wt. % or 5 to 20 wt. %) and can generally be made available as a separate valuable product through suitable processing.

Adjustment of the ratio of ethylene to acetic acid in the product stream of the oxidative catalytic dehydrogenation using MoVNbTeOₓ catalysts is possible in particular by adjusting the water partial pressure in the reactant, but in particular in the reactor outlet stream, the space velocity and the operating pressure, but only within certain limits (see e.g. WO 2018/115416 A1, EP 3 519 377 B1 and WO 2019/243480 A1). The invention, however, allows the free adjustment of other conditions within the scope explained. A minimum water content is advantageous in order to ensure stable catalyst performance (see e.g. WO 2018/115418 A1, EP 3 558 910 B1 and WO 2019/243480 A1). With the aid of a specially adapted reactor design in combination with optimized operating conditions (e.g. space velocity) and/or linear velocity, an increase in the ethylene yield can be achieved (see U.S. Pat. No. 10,017,432 B2 and U.S. Pat. No. 9,963,412 B2). However, even here the acetic acid content in the product is still too high to discard it. Therefore, the use of acetic acid as a product is indispensable and it is necessary to provide quantities of acetic acid according to requirements.

The use of oxidative catalytic dehydrogenation is mostly limited to small to medium plant capacities due to the coupled production and requires appropriate utilization of the acetic acid produced. On the other hand, in contrast to the steam cracker, oxidative catalytic dehydrogenation produces (essentially) only ethylene and acetic acid as products. The requirement for suitable utilization of other product fractions and the corresponding equipment in the fractionation part is thus also eliminated. The invention now deliberately makes use of the formation of acetic acid during oxidative catalytic dehydrogenation and advantageously provides both co-products.

A plant for oxidative catalytic dehydrogenation typically comprises the substantial process steps already described in relation to FIG. 1, which can be arranged in a suitable manner. In addition to the oxidative catalytic dehydrogenation, a condensate separation 120, an acetic acid treatment 130, a compression 140 and a carbon dioxide removal 160 (in particular amine washing and possibly lye washing for fine cleaning) as well as an ethane-ethylene separation 190 can. Furthermore, if required, further process steps, such as demethanization 180, as well as trace removal or raw gas treatment (also with the addition of hydrogen and oxygen, see e.g. WO 2020/187572 A 1), as well as selective hydrogenation of hydrocarbons with two carbon atoms, can be used at a suitable location. Separation of hydrocarbons with two and three carbon atoms can also be used, which can typically be arranged upstream of the catalytic oxidative dehydrogenation in order to separate heavier components from the ODHE feed mixture.

Of particular importance in the context of the invention is the acetic acid preparation. As mentioned at the outset, acetic acid is a component of the condensate phase. According to the prior art, the acetic acid is advantageously extracted from the condensate phase. Organic solvents, particularly those from the ether group, such as methyl tert-butyl ether, or the ester group, such as ethyl acetate, are suitable as extraction agents. The acetic acid is then separated by distillation from the organic extract phase obtained in this way, the extractant being recovered for further extraction cycles. If necessary, the acetic acid obtained in this way can be subjected once again to a fine distillation until the desired purity (e.g. glacial acetic acid) is achieved. The aqueous raffinate phase, which also arises during the extraction step, can also be subjected to distillative processing in order to recover the extraction agent contained in the raffinate phase or to obtain wastewater with as little pollution as possible. Through acetic acid processing, acetic acid can be obtained in particular in a purity of more than 95%, more than 98% or more than 99% and/or in glacial acetic acid quality, i.e. in a purity of at least 99.8%, and used for subsequent processes.

The preparation of acetic acid by other processes is basically well known and technically established (C. Le Berre, P. Serp, P. Kalck, G. P. Torrence, “Acetic Acid” in Ullmann's Encyclopedia of Industrial Chemistry 2014). In this case, the traditional and particularly important route of the reaction of methanol with carbon monoxide is used. Liquid phase oxidations of e.g. butane, naphtha or acetaldehyde are also known. The cited article also mentions approaches for the catalytic oxidation of ethylene, particularly highlighting the influence of Pd addition to the catalyst.

Oxidative processes starting from ethane using suitable mixed metal oxide catalysts (based on Mo, V and/or Nb) have also already been developed and commercialized. Special modifiers are used in this case. For example, U.S. Pat. No. 5,907,056 proposes A P, B, Hf, Te and As modifiers and assumes exclusively ethane as feedstock for oxidative catalytic dehydrogenation. The examples in this document achieve ethane yields of up to about 66% at ethylene to acetic acid ratios between about 1:1 and 5:1. Only for entries 7 and 8 in Table 3 of the document are ethylene to acetic acid ratios of approximately 1.9:1 and 5:1, respectively, achieved, but a P-modified catalyst is used. Although U.S. Pat. No. 6,258,992 A discloses feed streams that may contain alkenes, such as ethylene, in addition to alkanes, the catalyst disclosed therein necessarily always also contains a sulfur species. The examples in the document are limited to yields of less than 20% (starting from pure ethane), with the resulting ratios of ethylene to acetic acid being between 1:1 and 1:7. According to other applications, e.g. according to EP 1,140,355 B1, further promoters are added, in particular the (expensive) noble metal Pd, in order to increase the selectivity with respect to acetic acid. However, the examples in this document are limited to paraffins as the feed stream for oxidative catalytic dehydrogenation, and propane is also investigated in addition to ethane. U.S. Pat. No. 6,030,920 A also includes the addition of Pd to a MoVNbOₓ catalyst and is explicitly limited to the reaction of ethane as a feed stream for a corresponding selective oxidation.

In this case, processes that require both ethylene and acetic acid as reactants require both reactants in a defined ratio. This ratio of ethylene to acetic acid (on a molar basis) is, for example, in particular for the production of vinyl acetate monomer, 3:1 to 1:1, of ethyl acetate 3:1 to 1:1 and of ethylene vinyl acetate 12:1 to 8:1. Ethylene vinyl acetate can be obtained from vinyl acetate monomer by reaction with ethylene, which explains the very high ratio of ethylene to acetic acid, or the ratio is additionally determined by the degree of copolymerization between ethylene and vinyl acetate monomer.

Another process is the preparation of polyethylene terephthalate via terephthalic acid, which requires an ethylene to acetic acid ratio of 14:1 to 11:1 and requires acetic acid as a solvent. In principle, the acetic acid is recycled in this case, but due to the harsh reaction conditions during the catalytic oxidation of p-xylene (typically with atmospheric oxygen at 175 to 225° C. and 15 to 30 bara), a significant loss of solvent occurs. Losses amount to up to 0.05 tonnes of acetic acid per tonne of polyethylene terephthalate.

However, as mentioned, the conventional catalytic oxidative dehydrogenation of ethane starting from ethane can provide ethylene and acetic acid only in a certain ratio, which can be influenced only to a limited extent by the choice of process conditions, in particular the water content in the reaction feed, the space velocity and the operating pressure and, if necessary, a special reactor design. In particular, a high acetic acid content—as required in particular for the downstream products vinyl acetate monomer or ethyl acetate—cannot be achieved in this way. This means that more ethylene must be produced than is actually needed, which is not always desirable, especially if there is no further demand for ethylene at a corresponding location or in a corresponding compound.

Alternatively, according to the prior art, acetic acid can be purchased or prepared using other processes, but this involves additional effort.

In principle, an increase in the acetic acid content towards an ethylene to acetic acid ratio of significantly less than 5:1 (preferably 3:1 or less) is conceivable. However, this will not be possible without a significant increase in the pressure design of the reactor and in addition a reduction in the plant capacity in order to meet the increased safety requirements resulting from an increased oxygen demand and the significantly increased pressure. This means that a significantly smaller molar product ratio of ethylene to acetic acid than 5:1 cannot be achieved with a reactor design with which a range of ethylene to acetic acid of approximately 5:1 to 10:1 can be easily adjusted. Overall, this leads to significantly higher complexity and thus significantly higher costs, which would call into question the economic viability.

The invention therefore discloses a solution for being able to adjust the ratio of ethylene to acetic acid as required and flexibly with a single technically established reactor design, in particular for an increased demand for acetic acid. Embodiments of the invention can be used in addition to already known measures, such as a change in the water content in the reaction feed, the space velocity, the linear velocity or the operating pressure of the oxidative catalytic dehydrogenation, and, in addition to such measures, a simple adaptation to a different required ratio of ethylene to acetic acid, in particular by adapting the ethylene recycling with otherwise identical apparatus requirements and operating conditions.

The percentage of recycled ethylene (“R_C2H4”), hereinafter also referred to as PRE value, of the total amount of recycled hydrocarbons with two carbon atoms (“RG”), i.e. the sum of ethane and ethylene, each in mol or kmol, can be expressed as follows:

PRE=n(R_C2H4)/n(R_G)×100%

Under the theoretical assumptions that all ethane in the product mixture not reacted in the oxidative catalytic dehydrogenation (“P_C2H6”) is recycled again to the oxidative catalytic dehydrogenation (technically based losses, e.g. in purge streams or residual contents in other process streams are thus neglected) and the recyclate does not contain any other components, such as small amounts of higher hydrocarbons and/or carbon monoxide, the following applies, where the indication “R_C2H6” stands for the recycled ethane:

n(R_G)=n(R_C2H6)+n(R_C2H4)=n(P_C2H6)+n(R_C2H4)

Assuming a typical ethane yield in oxidative catalytic dehydrogenation of 50% (technically usual values are in the range of approx. 40 to 60%), the values given in Table 1 thus result. In this case, the value n(P_C2H4) takes into account only the ethylene formed in the oxidative catalytic dehydrogenation of ethane, and does not include the unreacted ethylene. This value is therefore only for orientation and cannot be measured separately in reality. However, according to the invention, n(R_C2H4) is always smaller than n(P_C2H4). The value n(F_C2HG6) indicates the ethane content of the feed mixture.

TABLE 1

| | | Ethane to | | | | | |
|---|---|---|---|---|---|---|---|
| | Ethane | ethylene | | | | | |
| n(F_C2H6) | yield | selectivity | n(R_C2H6) | n(P_C2H4) | n(R_C2H4) | | PRE |
| 100 kmol | 40% | 80% | 60.0 kmol | 32.0 kmol | 1.0 | kmol | 1.64% |
| 100 kmol | 40% | 85% | 60.0 kmol | 34.0 kmol | 1.0 | kmol | |
| 100 kmol | 40% | 90% | 60.0 kmol | 36.0 kmol | 1.0 | kmol | |
| 100 kmol | 40% | 80% | 60.0 kmol | 32.0 kmol | 2.5 | kmol | 4.00% |
| 100 kmol | 40% | 85% | 60.0 kmol | 34.0 kmol | 2.5 | kmol | |
| 100 kmol | 40% | 90% | 60.0 kmol | 36.0 kmol | 2.5 | kmol | |
| 100 kmol | 40% | 80% | 60.0 kmol | 32.0 kmol | 5.0 | kmol | 7.69% |
| 100 kmol | 40% | 85% | 60.0 kmol | 34.0 kmol | 5.0 | kmol | |
| 100 kmol | 40% | 90% | 60.0 kmol | 36.0 kmol | 5.0 | kmol | |
| 100 kmol | 40% | 80% | 60.0 kmol | 32.0 kmol | 10.0 | kmol | 14.29% |
| 100 kmol | 40% | 85% | 60.0 kmol | 34.0 kmol | 10.0 | kmol | |
| 100 kmol | 40% | 90% | 60.0 kmol | 36.0 kmol | 10.0 | kmol | |
| 100 kmol | 40% | 80% | 60.0 kmol | 32.0 kmol | 15.0 | kmol | 20.00% |
| 100 kmol | 40% | 85% | 60.0 kmol | 34.0 kmol | 15.0 | kmol | |
| 100 kmol | 40% | 90% | 60.0 kmol | 36.0 kmol | 15.0 | kmol | |
| 100 kmol | 40% | 80% | 60.0 kmol | 32.0 kmol | 20.0 | kmol | 25.00% |
| 100 kmol | 40% | 85% | 60.0 kmol | 34.0 kmol | 20.0 | kmol | |
| 100 kmol | 40% | 90% | 60.0 kmol | 36.0 kmol | 20.0 | kmol | |
| 100 kmol | 50% | 80% | 50.0 kmol | 40.0 kmol | 1.0 | kmol | 1.96% |
| 100 kmol | 50% | 85% | 50.0 kmol | 42.5 kmol | 1.0 | kmol | |
| 100 kmol | 50% | 90% | 50.0 kmol | 45.0 kmol | 1.0 | kmol | |
| 100 kmol | 50% | 80% | 50.0 kmol | 40.0 kmol | 2.5 | kmol | 4.76% |
| 100 kmol | 50% | 85% | 50.0 kmol | 42.5 kmol | 2.5 | kmol | |
| 100 kmol | 50% | 90% | 50.0 kmol | 45.0 kmol | 2.5 | kmol | |
| 100 kmol | 50% | 80% | 50.0 kmol | 40.0 kmol | 5.0 | kmol | 9.09% |
| 100 kmol | 50% | 85% | 50.0 kmol | 42.5 kmol | 5.0 | kmol | |
| 100 kmol | 50% | 90% | 50.0 kmol | 45.0 kmol | 5.0 | kmol | |
| 100 kmol | 50% | 80% | 50.0 kmol | 40.0 kmol | 10.0 | kmol | 16.67% |
| 100 kmol | 50% | 85% | 50.0 kmol | 42.5 kmol | 10.0 | kmol | |
| 100 kmol | 50% | 90% | 50.0 kmol | 45.0 kmol | 10.0 | kmol | |
| 100 kmol | 50% | 80% | 50.0 kmol | 40.0 kmol | 15.0 | kmol | 23.08% |
| 100 kmol | 50% | 85% | 50.0 kmol | 42.5 kmol | 15.0 | kmol | |
| 100 kmol | 50% | 90% | 50.0 kmol | 45.0 kmol | 15.0 | kmol | |
| 100 kmol | 50% | 80% | 50.0 kmol | 40.0 kmol | 20.0 | kmol | 28.57% |
| 100 kmol | 50% | 85% | 50.0 kmol | 42.5 kmol | 20.0 | kmol | |
| 100 kmol | 50% | 90% | 50.0 kmol | 45.0 kmol | 20.0 | kmol | |
| 100 kmol | 60% | 80% | 40.0 kmol | 48.0 kmol | 1.0 | kmol | 2.44% |
| 100 kmol | 60% | 85% | 40.0 kmol | 51.0 kmol | 1.0 | kmol | |
| 100 kmol | 60% | 90% | 40.0 kmol | 54.0 kmol | 1.0 | kmol | |
| 100 kmol | 60% | 80% | 40.0 kmol | 48.0 kmol | 2.5 | kmol | 5.88% |
| 100 kmol | 60% | 85% | 40.0 kmol | 51.0 kmol | 2.5 | kmol | |
| 100 kmol | 60% | 90% | 40.0 kmol | 54.0 kmol | 2.5 | kmol | |
| 100 kmol | 60% | 80% | 40.0 kmol | 48.0 kmol | 5.0 | kmol | 11.11% |
| 100 kmol | 60% | 85% | 40.0 kmol | 51.0 kmol | 5.0 | kmol | |
| 100 kmol | 60% | 90% | 40.0 kmol | 54.0 kmol | 5.0 | kmol | |
| 100 kmol | 60% | 80% | 40.0 kmol | 48.0 kmol | 10.0 | kmol | 20.00% |
| 100 kmol | 60% | 85% | 40.0 kmol | 51.0 kmol | 10.0 | kmol | |
| 100 kmol | 60% | 90% | 40.0 kmol | 54.0 kmol | 10.0 | kmol | |
| 100 kmol | 60% | 80% | 40.0 kmol | 48.0 kmol | 15.0 | kmol | 27.27% |
| 100 kmol | 60% | 85% | 40.0 kmol | 51.0 kmol | 15.0 | kmol | |
| 100 kmol | 60% | 90% | 40.0 kmol | 54.0 kmol | 15.0 | kmol | |
| 100 kmol | 60% | 80% | 40.0 kmol | 48.0 kmol | 20.0 | kmol | 33.33% |
| 100 kmol | 60% | 85% | 40.0 kmol | 51.0 kmol | 20.0 | kmol | |
| 100 kmol | 60% | 90% | 40.0 kmol | 54.0 kmol | 20.0 | kmol | |

Due to the presence of small amounts of other components, such as higher hydrocarbons and/or carbon monoxide, the actual PRE value is somewhat lower in reality. Since ethylene is also used as a valuable product and in particular demand-based ratios of ethylene to acetic acid are to be achieved, particularly advantageous PRE values are in the range already mentioned above. In this way, in particular the advantageous ratios of ethylene to acetic acid mentioned above can be achieved for a process downstream of the oxidative catalytic dehydrogenation (i.e. without taking into account the ethylene which is recycled).

In a test plant, reactions of ethane and ethane-ethylene feedstocks were investigated under conditions of oxidative catalytic dehydrogenation. The corresponding test reactor is designed as a double tube and has a usable length of 0.9 m and an inner diameter of the reaction chamber of 10 mm. Heating or cooling is carried out with the aid of a thermal oil bath, the thermal oil being pumped through the outside of the reactor and thus heating or simultaneously cooling the interior/reaction zone (the reaction is an exothermic reaction). Due to the very good heat transfer, the oil bath temperature corresponds to the inlet temperature of the gas onto the catalyst bed (and has also been proven by measurements) and is therefore the reaction temperature. The test conditions and the results obtained thereby are shown in Table 2.

It can be seen that the selectivity shifts significantly towards acetic acid when ethylene is present in the reaction feed.

TABLE 2
Test conditions and results of the oxidative reaction
of ethane and an ethane-ethylene mixture.

| | | |
|---|---|---|
| Unit | Experiment 1 | Experiment 2 |

Experimental conditions

| | | | |
|---|---|---|---|
| Hydrocarbon | (mmol/h)_{KW}/kg_{Kat} | 26.7 | 23.0 |
| pollution | | | |
| Fraction of | % | 0 | 47.7 |
| ethylene in | | | |
| hydrocarbon | | | |
| consumption | | | |
| Ratio of water to | mol/mol | 1.18 | 0.39 |
| hydrocarbon | | | |
| feedstock | | | |
| Operating | bara | 4.9 | 3.1 |
| pressure | | | |
| Reaction | °C | 334 | 317 |
| temperature | | | |

Results

| | | | |
|---|---|---|---|
| Ethane yield | % | 55 | 42 |
| Ethylene | % | 74.5 | 51.9 |
| selectivity | | | |
| Acetic acid | % | 20.9 | 36.5 |
| selectivity | | | |
| Selectivity to | % | 4.6 | 11.4 |
| carbon oxides | | | |
| Product ratio | mol/mol | 3.6 | 1.4 |
| ethylene:acetic | | | |
| acid | | | |

The invention, in its embodiments, enables in particular a needs-based optimization and adaptation of the ethylene and acetic acid capacity beyond the usual limitations when using a pure ethane feed. A flexible adjustment of the ratio of ethylene to acetic acid is possible, especially towards high acetic acid contents. In particular, this is also possible beyond a ratio that can be achieved solely by varying the process parameters of the oxidative catalytic dehydrogenation.

The invention thus enables an advantageous use of catalytic oxidative dehydrogenation even for higher acetic acid capacities, regardless of the specific ethylene requirement. This eliminates the limitations that previously remained due to coupled production, since the ratio of ethylene to acetic acid can be freely adjusted within a wide range without the need for a special reactor design (e.g. adapted space or linear velocity). Rather, established technical reactor designs can continue to be used.

In embodiments of the invention, the ratio of ethylene to acetic acid can be quickly and flexibly adapted to changing requirements. Only a small additional effort is required for product purification by joint use of certain process steps (e.g. condensate separation and fractionation part). In particular, no additional processes for acetic acid preparation, or purchasing, are required. An additional feed for acetic acid preparation is not necessary. Furthermore, there is no need to use expensive precious metal additives (especially Pd) to increase the acetic acid selectivity. The catalyst is a conventional catalyst, in particular a mixed metal oxide catalyst.

Depending on the embodiment, less effort is required in the implementation of the ethane-ethylene separation or the C2 splitter, since complete ethylene separation is not necessary. Overall, the effort required is lower than with a steam cracker, and no significant amounts of by-products are formed (e.g. no or very little formation of methane, only traces of higher hydrocarbons). The overall process according to embodiments of the invention is therefore highly selective with respect to acetic acid and ethylene.

1. A process for preparing ethylene and acetic acid, comprising:
subjecting ethane and oxygen in a feed mixture to oxidative catalytic dehydrogenation using a catalyst containing at least molybdenum, vanadium and niobium to obtain a product stream containing ethylene, acetic acid and further components,
wherein the feed mixture has an ethylene content of 0.25 to 30 mol. %, and at least a portion of the ethylene content of the feed mixture of the oxidative catalytic dehydrogenation is formed by ethylene which is contained in the product stream and is recycled to the oxidative catalytic dehydrogenation;
subjecting the product stream or a portion thereof to a primary treatment, from which a subsequent mixture is taken, the subsequent mixture being depleted in carbon dioxide and water compared to the product stream;
feeding the subsequent mixture or a portion thereof as a low-temperature separation feed to a low-temperature separation which comprises a demethanization, wherein:
a heavy fraction and a light fraction are formed as an overhead fraction in the demethanization;
the heavy fraction is enriched in hydrocarbons with two carbon atoms, in particular ethane and ethylene in total, compared with the subsequent mixture, and is depleted in components boiling lower than ethylene; and
the light fraction is depleted in hydrocarbons with two carbon atoms compared to the subsequent mixture and enriched in components boiling lower than ethylene and has an ethylene content of 1 to 90 mol or volume percent; and
recycling the light fraction or a portion thereof to the oxidative dehydrogenation.

2. The process according to claim 1, wherein, in the light fraction, a content of carbon monoxide is up to 75%, a content of methane is up to 25%, and an oxygen content is less than 5%, wherein the percentages represent mole or volume percent.

3. The process according to claim 1, further comprising recycling a proportion of 1% to 35% of the total ethylene contained in the product stream of the oxidative catalytic dehydrogenation to the oxidative catalytic dehydrogenation.

4. The process according to claim 3, wherein the recycled ethylene is recycled to the oxidative catalytic dehydrogenation with ethane contained in the product mixture of the oxidative catalytic dehydrogenation.

5. The process according to claim 1, wherein:
the further components of the product mixture of the oxidative catalytic dehydrogenation comprise unreacted ethane, compounds boiling lower than ethylene, including carbon monoxide, as well as carbon dioxide and water; and
the subsequent mixture contains at least a portion of the ethane, the ethylene and the compounds boiling lower than ethylene from the product mixture.

6. The process according to claim 5, wherein the primary processing comprises a raw gas treatment, a carbon dioxide removal, and a drying, wherein a portion of a gas mixture taken from the raw gas treatment is discharged from the primary processing upstream of the carbon dioxide removal and drying and is recycled to the oxidative catalytic dehydrogenation.

7. The process according to claim 1, wherein:
at least a portion of the low-temperature separation feed is fed as an ethane-ethylene separation feed to the ethane-ethylene separation;
in the ethane-ethylene separation, a heavy fraction (C2H6) enriched in ethane compared to the subsequent mixture and depleted in ethylene and the components boiling lower than ethylene, and a light fraction (C2H6) depleted in ethane compared to the subsequent mixture and enriched in ethylene and the components boiling lower than ethylene are formed; and
the light fraction or a portion thereof is fed as a demethanization feed to the demethanization.

8. The process according to claim 1, further comprising:
selecting one or more fractions selected from list consisting of the bottom fraction formed in the demethanization, the overhead fraction (C2H4) formed in the ethane-ethylene separation, and the bottom fraction (C2H6) formed in the ethane-ethylene separation; and
recycling, at least in part, the selected fraction(s) to the oxidative dehydrogenation.

9. The process according to claim 8, further comprising:
selecting one or more further fractions selected from list consisting of the overhead fraction formed in the demethanization, the bottom fraction formed in the demethanization, the overhead fraction (C2H4) formed in the ethane-ethylene separation, and the bottom fraction (C2H6) formed in the ethane-ethylene separation; and
forming the one or more further fractions as a fraction containing ethane and ethylene.

10. The process according to claim 1, further comprising:
taking a side stream containing ethane and ethylene from the ethane-ethylene separation; and
at least partially recycling the side stream to the oxidative dehydrogenation.

11. The process according to claim 1, further comprising:
transferring at least a portion of the ethylene contained in the product mixture to an open refrigerant circuit; and
discharging at least a portion of the ethylene from the open refrigerant circuit and recycling to the oxidative catalytic dehydrogenation to provide at least a portion of the ethylene portion of the feed mixture.

12. The process according to claim 1, further comprising subjecting at least one material stream recycled to the oxidative catalytic dehydrogenation to provide at least a portion of the ethylene content of the feed mixture to processing.

13. A process for preparing a target compound using ethylene and acetic acid, comprising utilizing the process according to claim 1 to provide at least a portion of the ethylene and the acetic acid.

14. (canceled)

15. The process according to claim 1, wherein the catalyst further contains tellurium as a mixed oxide.

16. The process according to claim 2, further comprising recycling a proportion of 1% to 35% of the total ethylene contained in the product stream of the oxidative catalytic dehydrogenation to the oxidative catalytic dehydrogenation.

17. The process according to claim 16, further comprising recycling the recycled ethylene to the oxidative catalytic dehydrogenation with ethane contained in the product mixture of the oxidative catalytic dehydrogenation.

18. The process according to claim 1, wherein, in the light fraction, a content of carbon monoxide is up to 75%, a content of methane is up to 25%, and an oxygen content is below the oxygen limit concentration, wherein the percentages represent mole or volume percent.

19. The process according to claim 18, wherein the oxygen content is less than 5%.
